(19) United States
(12) Patent Application Publication
(10) Pub. No.: US 2009/0047241 A1
(43) Pub. Date: Feb. 19, 2009

(54) IL-12 FACILITATES BOTH THE RECOVERY OF ENDOGENOUS HEMATOPOIESIS AND THE ENGRAFTMENT OF STEM CELLS AFTER IONIZING RADIATION

(75) Inventors: Yi Zhao, South Pasadena, CA (US); Tingchao Chen, Monterey Park, CA (US); Yuxia Zhan, Monterey Park, CA (US)

Correspondence Address: HOGAN & HARTSON L.L.P., 1999 AVENUE OF THE STARS, SUITE 1400, LOS ANGELES, CA, 90067, US

(73) Assignee: University of Southern California, Los Angeles, CA (US)

(21) Appl. No.: 12/142,146

(22) Filed: Jun. 19, 2008

Related U.S. Application Data
(60) Provisional application No. 60/945,280, filed on Jun. 20, 2007.

(51) Int. Cl. A61K 38/20; C07K 14/54; A61P 19/08

(52) U.S. Cl. 424/85.2; 530/351

(57) ABSTRACT

The invention provides compositions and methods for promoting the recovery of bone marrow micro-environment after disease- or treatment-induced damage.

The present application claims the benefit of the filing date of U.S. Provisional Application No. 60/945,280, filed Jun. 20, 2007, the disclosure of which is incorporated herein by reference in its entirety.

### FIELD OF THE INVENTION

The present invention relates in general to hematopoiesis and stem cell transplantation. More specifically, the invention provides compositions and methods for promoting the recovery of patients' bone marrow micro-environment after disease- or treatment-induced damage.

### BACKGROUND OF THE INVENTION

Severe myelosuppression is a common side effect of radiotherapy or chemotherapy. Methods have been developed to protect patients by stimulating white blood cell or red blood cell recovery/production using growth factors such as G-CSF or EPO. However, there is no available means to stimulate the full lineage blood cell recovery from severe myelosuppression.

The hematopoietic stem cell (HSC) compartment resides in the bone marrow and produces full lineage blood cells throughout the lifespan. A functional hematopoietic system relies on HSC and their supporting microenvironment/niche. Via direct cell-cell interaction or soluble factors produced locally or systemically, the microenvironment regulates the quiescence, apoptosis, self-renewal, proliferation and differentiation of stem cells [1-4]. The mechanism for this complicated regulation remains largely unknown. Accumulated evidence has shown that, as a niche component, osteoblasts play a pivotal role in HSC regulation [5-8]. Recently, using different approaches, two groups demonstrated that sinusoidal endothelial cells are another important element of the niche [9,10]. It has been shown that disruption of osteoblasts or sinusoidal endothelial cells results in the hematopoiesis dysfunction. On the other hand, stimulation of the osteoblasts or sinusoil endothelial cells leads to increase of HSC numbers [6,8,9] or recovery of HSC from myelosuppession [10].

Bone marrow is the most sensitive organ to ionizing radiation and/or chemotherapeutic drugs during cancer therapy. Myelosuppression and hematopoietic dysfunction are the most common clinical complications following radio-/chemo-therapy. These physical or chemical insults can damage hematopoiesis by targeting either HSC directly or alternatively their microenvironment or both. Obviously, it is important in cancer treatment to promote the recovery of hematopoiesis from myelosuppression.

Consequently, a number of cytokines and combinations of cytokines have been studied using lethally irradiated animals [11-18]. Most of the studies observed the animal survival for 30 days after lethal radiation. With a combination of 5 anti-apoptotic factors, Herodin et al. reported 50% long-term survival (360 days) of the rescued animals. The receptors of the factors used in the previous studies, such as SCF, SDF-1, TPO, and Flt-3 ligand, are expressed on the HSC/progenitor cells. The protection from these factors may be a direct effect on the stem/progenitor cells. However, in the GI system, the FGF-2 mediated radioprotective effect on intestinal crypt stem cells is via the protection of the endothelial cells adjacent to crypt stem cells, since FGF-2 receptor could only be detected on endothelial cells [19]. Since HSC function is closely related to the structure and function of microenvironment, the recovery of hematopoiesis from lethal radiation requires the recovery of both the microenvironment and the HSC.

Interleukin-12 (IL-12) is a heterodimeric pro-inflammatory cytokine that regulates the activity of cells involved in the immune response [20-22]. It stimulates the production of IFN-y from natural killer cells and T cells, favors the differentiation of T helper 1 cells, and forms a link between innate resistance and adaptive immunity. Under in vitro conditions, IL-12 can stimulate the hematopoiesis synergistically with IL-3 and SCF [23,24]. It has been reported that, at the price of sensitizing the GI system, IL-12 can protect the bone marrow from lethal irradiation [15]. However, in contrast to other radioprotective factors that may stimulate tumor cell proliferation or angiogenesis, such as SCF, SDF-1 and FGF-1[25-28], IL-12 inhibits tumor cell growth [29] and is anti-angiogenic [30,31].

### SUMMARY OF THE INVENTION

In accordance with one embodiment, the invention relates to compositions comprising IL-12 that promote the recovery of hematopoeisis in damaged bone marrow cells.

In accordance with another embodiment, the invention relates to compositions comprising IL-12 that protect bone marrow from radiation and/or chemotherapy.

In accordance with a related embodiment, the invention relates to compositions comprising IL-12 that are useful as an hematopoietic-protecting agent.

In accordance with yet another embodiment, the invention relates to compositions comprising IL-12 that are useful in the treatment of cancer.

In one embodiment, the invention relates to methods of using compositions comprising IL-12 to promote the recovery of hematopoeisis in damaged bone marrow cells.

In another embodiment, the invention relates to methods of using compositions comprising IL-12 to protect bone marrow from radiation and/or chemotherapy.

In a related embodiment, the invention relates to methods of using compositions comprising IL-12 as an hematopoietic-protecting agent.

In yet another embodiment, the invention relates to methods of using compositions comprising IL-12 to treat cancer.

In a further embodiment, the invention relates to compositions comprising IL-12 that protect the bone marrow microenvironment from the damage caused by radiation and/or chemotherapy.

In another further embodiment, the invention relates to methods of using compositions comprising IL-12 to protect the bone marrow microenvironment from the damage caused by radiation and/or chemotherapy.

In a further related embodiment, the invention provides methods to facilitate the engraftment of stem cell transplantation.

The above-mentioned and other features of this invention and the manner of obtaining and using them will become more apparent, and will be best understood, by reference to the following description, taken in conjunction with the accompanying drawings. The drawings depict only typical embodiments of the invention and do not therefore limit its scope.

### BRIEF DESCRIPTION OF THE FIGURES

FIG. 1. Effect of IL-12 mediated radioprotection. (a) Animal survival rate with IL-12 administration (100 ng/mouse) at 24 hrs before or 1 hr after lethal dose (10 Gy) radiation. Mice in the control group received PBS buffer. Results are summarized from 5 individual experiments with 5-12 mice in each group. IL-12 administration effectively protected/rescued the animals in the long-term (p<0.001 between IL-12 treated and control group; p<0.05 between IL-12 administrated 24 hrs before and 1 hr after radiation). (b) Pathological changes in the bone marrow of animals in treated and control groups. Femurs from IL-12 treated or control animals were isolated at different times post irradiation for pathology analysis. Hematopoiesis recovery was observed in IL-12 treated animals (arrows point to the mononuclear colonies, Hematoxylin & Eosin staining, 200× magnifications). d: days post radiation. Representative sections were shown from two experiments with 3 mice in each group.

FIG. 2. Pathological changes in the small intestine. Small intestines were isolated from animals in IL-12 treated or control groups at day 3 post irradiation. After fixation and HE staining, sections were examined microscopically (200× magnification). Different experimental conditions were tested: animals received no treatment or 10 Gy (one dose) radiation and IL-12 (100 ng/mouse). (a) Animals received 16 Gy (one dose) radiation or 16 Gy radiation and either IL-12 at 100 ng/mouse or 1000 ng/mouse. (b) Representative sections were shown from two experiments with 3 mice in each group.

FIG. 3. The effects of IL-12 treatment on peripheral blood cell count (PBCC). IL-12 promotes the recovery of PBCC after lethal dose irradiation (a) (split dose, 5 Gy for each dose, 3 hrs apart, n=5) and attenuates the decline of PBCC after sublethal dose irradiation (b) (5 Gy, n=10). Mouse blood samples were collected at different indicated times from tail vein and blood cell counts were measured immediately. The Y axis is the percentage change of cell count for each blood cell subtype after irradiation relative to its baseline cell count before irradiation and IL-12 treatment (*: p<0.05; **: p<0.01). The solid lines and the broken lines represent blood cell counts from the IL-12 treated and the control group respectively; error bars represent SD. Bg: background.

FIG. 4. The effects of IL-12 treatment on different HSC subsets after lethal dose irradiation (split dose). Bone marrow cells were isolated at difference times post irradiation from IL-12 treated or control animals and examined in BMT (1×10⁷ cells for each recipient), CFU-S₁₂ (2×10⁵ cells for each recipient) and CFC assay (2×10⁵ cells for each plate). (a) Recovery of LTR HSC activity in BMT (Summary of 2 experiments, the number above the bar is the number of survivors out of the total number of experimental mice). (b) Recovery of CFU-S₁₂ cell activity. The inserted picture showed CFU-S₁₂ colonies derived from donor cells isolated from IL-12 treated mice (summary of 2 experiments with total of 6 mice, WT: CFU-S₁₂ activities from mice without experimental treatments). (c) The recovery of progenitor cell activities detected via CFC assay. The solid line represented the change of CFC numbers in IL-12 treated mice, the dotted line was the CFC numbers from bone marrow cells isolated from mice without experimental treatment (no radiation, no IL-12), and there was no detectable CFC in the control mice (the dashed lines on the X axis). Summary of two experiments with a total of 6 mice. (d-f) IL-12 rescued bone marrow cells maintain LTR HSC characters. 6 months after radiation, bone marrow cells were isolated from IL-12 rescued mice and transplanted to lethally irradiated recipients. 4 months after BMT, blood was collected and stained with anti-Ly5.2, anti-Mac-1 and anti-Gr-1, or with anti-Ly5.2, anti-CD3 and anti-B220 antibodies. Donor cell (from IL-12 rescued C57BL/Ly5.2 mice) derived reconstitution is demonstrated in the peripheral blood (d). IL-12 rescued bone marrow cells can differentiate into both myeloid (e) and lymphoid (f) cells (n=5, mean±SD). IR: irradiation.

FIG. 5. No detectable IL-12 receptor expression on LTR HSC.

(a) Lin⁻ cells were stained with FITC-conjugated anti-Sca-1 and APC-conjugated anti-c-kit antibodies for FACS analysis; (b) Lin⁻Sca⁺c-kit⁺ Cells (in gate R2) were reanalyzed for IL-12 receptor expression by staining with PE-conjugated anti-IL-12 receptor β2 chain antibody.

(c) cDNAs from 4 individual sorted samples with 5000 of Lin⁻Sca⁺c-kit⁺CD34⁻ cells in each were analyzed for the expression of IL-12 receptor and CD3e, β-actin expression was used as internal control. The first lane was no RT control and the second lane was RT-PCR from bone marrow Lin⁻ cells as positive control for IL-12 receptor and CD3e. Lanes 1-4 were results from 4 individual sorted samples.

FIG. 6. IL-12 treated bone marrow facilitated donor cell engraftment in BMT. (a) IL-12 treatment did not enhance the homing in BMT. After lethal dose irradiation, bone marrow cells (1×10⁶) from GFP transgenic mice were transplanted to IL-12 treated or control mice. At different times post irradiation, bone marrow cells were isolated from recipients to determine donor cell homing efficiency (Day 1, 24 hrs post irradiation) and proliferation (day 3, 5, 7 and 30 post radiation). The inserted figure showed the GFP cell number in recipient's bone marrow 24 hrs and 3 days post transplantation (**: p<0.01, n=3, error bars represents SD). (b) The combination of a non-effective dose of HSC (100 Lin⁻Sca-1⁺c-kit⁺ cells) and a non-effective dose of IL-12 (25 ng/mouse) rescued lethally irradiated animals. 24 hrs before irradiation, animals received either PBS or IL-12 (25 ng) injection. Immediately following irradiation, each group was given either 100 Lin⁻Sca-1⁺c-kit⁺ cells or mock treatment. Survival rate was determined (n=5).

FIG. 7. Effects of IL-12 on bone marrow cell subsets. (a) Representative FACS analysis for determining AnnexinV⁻ population with specific cell surface marker (Sca-1). 4 hours post irradiation, bone marrow cells from IL-12 treated and control mice were isolated and stained with Annexin V and anti-Sca-1 antibodies. (b) The proportion of AnnexinV⁻/Sca-1⁺ cell was significantly higher in IL-12 treated animals (19.2±3.7% versus 9.7±1.5%, p<0.05, n=3, representative of 3 individual experiments, mean±SD). However, there was no significant difference of AnnexinV⁻/c-kit⁺ cell number between IL-12 treated and control animals 4 hrs post radiation. (c) There were no detectable Lin⁻Sca-1⁺c-kit⁺ cells 24 hrs after irradiation in both IL-12 treated and control animals. (d) Sca-1⁺ cells were present at significantly higher level in IL-12 treated animals. Bone marrow cells from IL-12 treated and control animals were isolated, stained with anti-Sca-1 antibody and analyzed by FACS at different times post irradiation (**: p<0.01, n=3, representative of 3 experiments, mean±SD).

### DETAILED DESCRIPTION OF THE INVENTION

Bone marrow/hematopoietic stem cell transplantation has been widely applied to treat both malignant and non-malignant diseases. In most of the cases, conditional regimen, high dose of chemotherapy treatment or combination of chemotherapy and radiation, is required. The purpose of this regimen is to kill the malignant cells and/or eliminate the host immune-system resulted rejection to the donor cells. However, in this process, the recipients bone marrow are severe impaired, which leads to various side effects, including bone marrow failure. The other commonly seen complication in stem cell transplantation is the engraftment failure, meaning that the donor cells can not proliferate and repopulate the blood system after transplantation. Based on the statistical summary, the successful engraftment rate with allogeneic stem cell transplantation is about 40-60%. The HSC engraftment failure may result from various reasons. Besides the quantity and quality of transplanted stem cell, the bone marrow microenvironment plays key role in the success of the HSC transplantation.

When transplanted HSC home to bone marrow, cells in bone marrow microenvironment provide the support for the HSC growth and proliferation. It has been reported that transplantation of non-HSC bone marrow cells resulted in a better engraftment of HSC in transplantation. The inventors show that IL-12 can facilitate the HSC engraftment by protecting the bone marrow microenvironment from the damage of conditional regimen, which ultimately facilitates the engraftment of HSC.

Currently, there is no effective method to facilitate the engraftment of stem cells in the stem cell transplantation. To improve the success rate of stem cell transplantation, large efforts has been put into the stem cell ex vivo expansion. In animal model, it has been reported that co-transplantation of other bone marrow cellular components, such as mesenchymal stem cells, may increase the chance of success in stem cell transplantation. However, both methods are premature and require cellular component, which increase the difficulty for clinical application.

The inventors have provided a simple but effective method to facilitate the engraftment of stem cell transplantation. By injection of IL-12 before or after conditional regimen, the protective effects of IL-12 to the bone marrow non-HSC preserve the bone marrow microenvironment, which than provide the supporting system for the transplanted stem cells for their growth and proliferation and maturation. As shown below, with limited non-rescue stem cell number, IL-12 treated bone marrow can support the early engraftment of transplanted stem cells and resulted in the rescue of the lethally irradiated animals.

Injection of IL-12 can facilitate the engraftment of stem cells in stem cell transplantation. When neither non-rescue dose of HSC or non-rescue does of IL-12 can protect animals from lethal dose radiation (mimic the conditional regimen in stem cell transplantation, no animal survived), a combination of the two results in 80% of survival rate. Further more, with IL-12 treatment, the donor cells give earlier and better proliferation after transplantation.

To improve the success of stem cell transplantation, efforts have been put in developing the methods of stem cell in vitro expansion. Other methods, such as co-transplantation of other cellular components or using expanded stem cells have also been reported in animal studies. However, both methods are patient-specific, which needs to isolate cells from patient and treat the cells in vitro. All the procedures are complicated and expansive.

The potential of using IL-12 to protect and facilitate the hematopoiesis has been provided. The inventors show that at a proper dose level, IL-12 can protect the bone marrow hematopioetic system and promote engraftment in BMT without adversely affecting GI system. IL-12 may play its role by affecting the cells in bone marrow microenvironment.

The present invention relates to IL-12 treated, lethally or sublethally irradiated animals that were examined for the survival/life-span, bone marrow cell functional recovery and bone marrow transplantation effect using competitive transplantation, CFU-S₁₂, Colony Forming Cell (CFC), and apoptosis assay.

The invention shows that at a low dose (10 times lower than previously reported dose), 91.4% of lethally irradiated animals survived long-term without adversely affecting the gastro-intestine (GI) system. The reconstituted hematopoietic system was derived from long term repopulating hematopoietic stem cells (LTR HSC), which can reconstitute hematopoiesis both endogenously after lethal radiation and in secondary recipients by bone marrow transplantation (BMT). IL-12 significantly attenuated the decline of blood cell counts in sublethally irradiated animals. The stimulated hematopoiesis recovery resulted in a full lineage blood cell production, including white and red blood cells, and platelets. There was no detectable expression of IL-12 receptor on LTR HSC. In IL-12 treated animals, Sca-1⁺ cells were significantly higher than animals without IL-12 treatment.

The invention shows that IL-12 may be used as an anti-tumor, anti-angiogenic agent and that the hematopoietic-protecting effects to severe myelosuppression may have clinical significance in cancer treatment and BMT.

To practice methods relating to the administration of compositions comprising IL-12, compositions are formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates; and agents for the adjustment of tonicity such as sodium chloride or dextrose. pH can be adjusted with acids or bases, such as hydrochloric acid or sodium hydroxide. The parenteral preparation can be enclosed in ampoules, disposable syringes, or multiple dose vials made of glass or plastic.

In one embodiment, IL-12 compositions are prepared with carriers that will protect the compounds against rapid elimination from the body, such as a controlled release formulation, including implants and microencapsulated delivery systems. Compositions, may also comprise siRNAs conjugated to cationic polypeptides, amphipathic compounds, polycations, liposomes or PEGlyated liposomes. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art.

It is advantageous to formulate oral or parenteral compositions in dosage unit form for ease of administration and uniformity of dosage. “Dosage unit form,” as used herein, refers to physically discrete units suited as unitary dosages for the subject to be treated, each unit containing a predetermined quantity of an active compound calculated to produce the desired therapeutic effect in association with the required pharmaceutical carrier.

The dosage required for treating a subject depends on the choice of the route of administration, the nature of the formulation, the nature of the subject's illness, the subject's size, weight, surface area, age, and sex, other drugs being administered, and the judgment of the attending physician. Wide variations in the needed dosage are to be expected in view of the variety of compounds available and the different efficiencies of various routes of administration. For example, oral administration would be expected to require higher dosages than administration by intravenous injection. Variations in these dosage levels can be adjusted using standard empirical routines for optimization as is well understood in the art. Encapsulation of the compound in a suitable delivery vehicle (e.g., polymeric microparticles or implantable devices) may increase the efficiency of delivery, particularly for oral delivery.

The following examples are intended to illustrate, but not to limit, the scope of the invention. While such examples are typical of those that might be used, other procedures known to those skilled in the art may alternatively be utilized. Indeed, those of ordinary skill in the art can readily envision and produce further embodiments, based on the teachings herein, without undue experimentation.

### Materials and Methods

### Mice, Cytokine and Antibodies

6 to 8 weeks old C57BL/6J female mice were obtained from Jackson Laboratory (Bar Harbor, Me.) and maintained in the animal facility at University of Southern California (USC, Los Angeles, Calif.) as described previously [32]. The animal study protocol was approved by the University of Southern California Animal Care and Use Committee. Recombinant murine interleukin 12 (rmIL-12) was purchased either from R&D Systems, Inc (Minneapolis, Minn.), or PeproTech Inc (Rocky Hill, N.J.) and was dissolved in phosphate buffered saline (PBS) at 100 ng/μl stock concentration according to the manufacturer's recommendation, and stored at −70° C. Antibodies used in the study were purchased from BD Biosciences (San Jose, Calif.).

### Radioprotection Assay and Blood Cell Count Analysis

rmIL-12 was intravenously (i.v.) injected into mice before or after total body irradiation at the indicated time points. Mice in the control group received PBS. Mice were lethally irradiated with 10 Gy given in two fractions of 5 Gy 3 hours apart using dual, opposed sources of cesium 137 irradiator (Atomic Energy of Canada, Model: γ-cell 40) or one dose of radiation at 10 Gy.

In an experiment to study IL-12 effects to sublethally treated animals, 5 Gy was given to animals 24 hours after IL-12 treatment. Base line blood counts were collected before IL-12 injection. Then, at different times after radiation, blood cell counts were measured.

To determine the peripheral blood cell counts, 10 ul blood was collected from the tail vein, diluted in 490 ul PBS buffer, and then analyzed in a MASCOT Multispecies Hematology Systems (CDC Technologies, Oxford, Conn.).

### Pathology of Bone Marrow, Spleen and Small Intestine

At different days after lethal dose irradiation, femurs and small intestines were removed from IL-12 treated and control animals and fixed in 10% formalin buffer. The femurs were decalcified in Immunocal Formic Acid Bone Decalcifier (Decal Corporation, NY) for 2 hours. The decalcified femurs and small intestine were embedded in TissuePrep 2 paraffin wax for micro-section at 5 μm and routine Hematoxylin & Eosin staining was performed. Slides were examined under microscope (Nikon E CLPSE E 8000, Camera: Diagnostic Model 2.2.1 with Spot RT software V3.5).

### Long-Term HSCs Repopulation Assay

1) After IL-12 administration (100 ng/mouse, 24 hrs before radiation) and irradiation (10 G), bone marrow cells from donor mice (IL-12 treated C57BL/Ly5.2 mice) were isolated at different times post irradiation. Bone marrow cells were flushed out and the red blood cells were lysed as previously described. 1×10⁷ donor cells were transplanted to lethally irradiated recipients (C57BL/Ly5.1, 10 G) to determine the long-term repopulation activity. Mice were observed daily and surviving rate was recorded. There were 10 recipients for each time point.

2) After survival for 6 months after IL-12 treatment, bone marrow cells (C57BL/Ly5.2) were isolated and injected (1×10⁶) to lethally irradiated recipients (C57BL/Ly5.1 mice, 10 G). 4 months after the transplantation, peripheral blood was collected to determine the donor cell derived reconstitution (anti-CD45.2 antibody), donor cell differentiated lymphoid cells (T and B cell with anti-CD3 and anti-B220 antibody) and myeloid cells (macrophage and granulocyte cell with anti-CD11b and anti-Gr1 antibody) via FACS analysis. There were 5 mice in each group.

### Colony-Forming Units (CFU)-Spleen₁₂ Assay

24 hrs after intravenous injection of IL-12 (100 ng/mouse) or PBS, mice were lethally irradiated (10 Gy, one time exposure). At indicated time after radiation, 2×10⁵ bone marrow cells were collected from the IL-12 treated or control mice and injected to recipient mice, which had received lethal dose irradiation. 12 days after transplantation, recipient mice were sacrificed and the spleens were removed and fixed in Tellyesniczky's solution as described previously [35]. Colonies on the spleen surface were counted. There were three mice in each group. The experiment was repeated twice.

### Colony-Forming Cells (CFC) Assay

24 hrs after intravenous injection of IL-12 (100 ng/mouse) or PBS, mice were lethally irradiated (10 Gy, one time exposure). At indicated times after radiation, 2×10⁵ bone marrow cells were collected for the assay following the manufacturer's instruction (MethCult GF M3434, StemCell Technologies Inc, Vancouver, BC). 2×10⁴ bone marrow cells from wild type animals were used in the assay. There were three mice in each group; the experiment was repeated twice.

IL-12 receptor expression

Lin⁻Sca-1⁺C-kit⁺CD34⁻ cells were isolated as previous described [33]. In brief, lineage negative cells were stained with anti-Sca-1, anti-c-kit and anti-IL-12 receptor (β2 subunit) for FACS analysis to determine the expression of IL-12 receptor. To perform RT-PCR analysis of IL-12 receptor expression, total RNA was isolated from 4 individual samples with 5000 Lin⁻Sca-1⁺c-kit⁺CD34⁻ cells. First-strand cDNA was synthesized from this RNA following the manufacturer's directions using the SuperScript III First-strand Synthesis System (Invitrogen) using oligo dT primers. Real-time PCR was done using the Roche LightCycler. Crossing points (CPs) of real-time PCR curves were determined by the LightCycler 3.5 software using the second derivative maximum method. PCR reactions were performed using the QuantiTect SYBR Green PCR Master Mix (Qiagen), 0.5 uM each primer and 1 or 2 μl of first-strand reaction (total volume 21 μl). PCR was performed in the LightCycler with 15 min at 95° C. (hotstart) followed by 45 cycles of 15 sec at 94° C., 30 sec at 55° C. and 15 sec at 72° C. Melting curve analysis followed to ensure that there were no primer dimers present and that the correct specific product was made. Primer design was done using Primer 3. For each sample, primers for IL-12 receptor β2 subunit, CD3e and β-actin were used to detect the expression of each corresponding cDNA. Primers used in the reaction are as following:

IL-12 receptor β2 forward primer:
5′GCAAACAGCACTTGGGTAAA3′,
reverse primer:
5′TTCCTGTAGCTTGTGGATTGG3′;
CD3e forward primer:
5′GATGCGGTGGAACACTTTCT3′,
reverse primer:
5′ACTGTCCTCGACTTCCGAGA3′;
beta-actin forward primer:
5′ACTGGGACGACATGGAGAAG3′,
reverse primer:
5′ACCAGAGGCATACAGGGACA3′.

### Homing and Engraftment Assay

24 hrs after intravenous injection of IL-12 (100 ng/mouse) or PBS, mice were lethally irradiated (5 Gy×2, 3 hrs interval). Four days after radiation, bone marrow cells (1×10⁶) GFP (Green Fluorescence Protein) transgenic mice (no treatment) were isolated and intravenously transplanted into lethally irradiated mice treated as described above. At 1, 3, 5, 7 and 30 days post transplantation, mice were killed and bone marrow cells were isolated from for FACS analysis for the presence of GFP cells. There were three mice in each group; the experiment was repeated twice.

To determine the stem cell engraftment, the recipient mice received non-protective dose of IL-12 (25 ng/mouse) or PBS. 24 hrs after the treatment, mice received lethally dose radiation (5 Gy×2, 3 hrs interval). Then, the recipient mice were intravenously transplanted with 100 Lin⁻Sca-1⁺c-kit⁺ cells or PBS. The method of 100 Lin⁻Sca-1⁺c-kit⁺ cell purification was described in previous publication [32].

The surviving rate of the transplanted mice was observed daily for 30 days. The results were presented in a Kaplan-Meier plot. There were 5 mice in each group.

### Apoptosis Assay and Bone Marrow Cell Subtype Determination

Cell apoptosis was analyzed by the TACS Annexin V-FITC Apoptosis Detection Kit (R&D Systems, Minneapolis, Minn.). 3×10⁵ cells were resuspended in the pre-mixed reaction solution containing 1× Binding buffer, Propidium iodide (100 ug/ml), and FITC-conjugated Annexin V of 250 ng/ml. Labeled cells were analyzed by FACS and apoptotic cells were defined as propidium iodide negative and FITC positive cells.

Cell apoptosis was analyzed by the TACS Annexin V-FITC Apoptosis Detection Kit (R&D Systems, Minneapolis, Minn.). 24 hrs after intravenous injection of IL-12 (100 ng/mouse) or PBS, mice were lethally irradiated (5 Gy×2, 3 hrs interval). 4 hrs after the irradiation, bone marrow cells were isolated and stained with FITC-conjugated anti-Annexin V and PE-conjugated anti-Sca-1 or anti-c-kit antibodies.

### Statistic Analysis

We analyzed the data by unpaired t-test and Kaplan-Meier and Log-rank test for survival rate.

### Results

Low-Dose Il-12 can Rescue Animals from Lethal Dose Irradiation and Promote Bone Marrow Recovery without Adverse Effects on GI

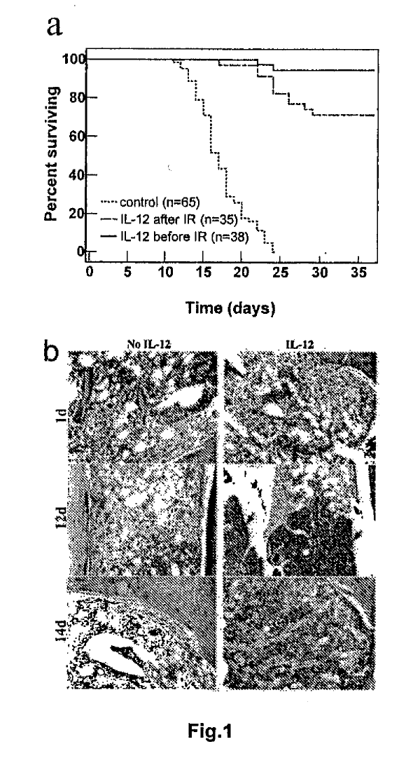

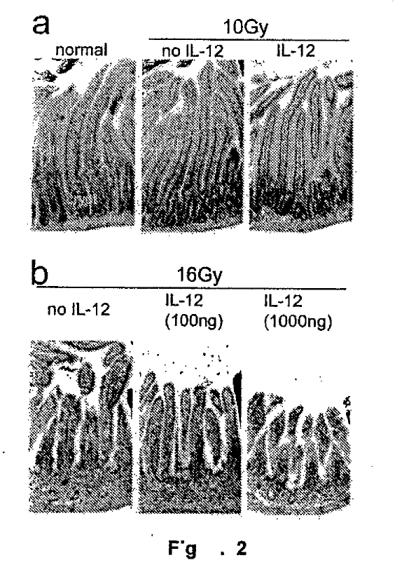

As previously reported, IL-12 mediated radioprotection were achieved with the cost of sensitizing GI system to radiation [15]. It has also been shown that IL-12 mediated GI sensitizing effect is related to IFN-y release, which is dose dependant event [15]. We reasoned that if the radioprotective effects could be achieved with lower dose of IL-12, we might be able to eliminate or significantly decrease the GI damage. First, we performed experiments to determine the optimal minimal dose for IL-12 mediated radioprotection. The tested dose ranged from 5 ng/mouse to 200 ng/mouse. IL-12 was administrated to animals after lethal dose of radiation (spit dose of 10 Gy) and the animal survivals were determined. 10 mice were tested for each dose of IL-12 administration. The minimal dose to achieve the maximum radioprotection (survival) is 100 ng/mouse (5 μg/Kg), which is 10 times lower than the dose used by Neta et. al. [15]. We next tested if the time of administration of IL-12 relative to the time of irradiation resulted in a difference in radioprotection. Varying the administration time of IL-12 relative to the time of irradiation (48, 36, 24 and 12 hrs before radiation; 1 hr, 12, 24 and 36 hrs post radiation), we found that the best rescue effect was achieved when IL-12 was given at 24 hrs before or 1 hr after irradiation. Animal survival rates after lethal irradiation are summarized in FIG. 1a: 91.4% and 75% of IL-12 treated mice survived long-term (more than one year) when IL-12 was administered at 24 hrs before or 1 hr after radiation, respectively. All the control mice died within 25 days after irradiation (p<0.001 for treated vs. control group; p<0.05 for IL-12 administrated 24 hrs before vs 1 hr after radiation). Since IL-12 treatment 24 hours before irradiation produced the best survival rate, all the subsequent experiments were performed using this protocol with 100 ng/mouse.

We then evaluated the bone marrow pathological changes of IL-12 treated and control animals at day 1, 3, 5, 7, 12 and 14 post radiation. As shown in FIG. 1b, the structure and the cellularity of bone marrows from animals in both groups were severely damaged. There was no significant difference between the two groups after the first 7 days post irradiation. However, at day 12, expansion of mononuclear cell colonies appeared in the IL-12 treated bone marrow, but not in the bone marrow of animals in the control group. At day 14, the bone marrow cellularity and structure of the IL-12 treated animals recovered almost to normal levels while the bone marrow in the control animals remained severely damaged.

Because of the report of Neta et al. [15], we determined if IL-12 sensitized the GI system to ionizing radiation under the conditions we used (100 ng versus 1000 ng/mouse). With 10 Gy radiation (a hematopoietic lethal dose), the structure and number of villi and crypt from both IL-12-treated (100 ng/mouse) and control mice remained intact (FIG. 2a). Even under the hyper-lethal dose of 16 Gy (a GI lethal dose), the level of GI damage in IL-12 treated mice was similar as the mice in control group (FIG. 2b). Also, with 10 Gy radiation, there is no significant body weight difference between the control and IL-12 treated groups in the first 14 days after radiation, suggesting that lower dose of IL-12 dose not sensitizing the GI system under a hematopoietic lethal dose of radiation (10 Gy). Thus, IL-12 did not show “radiation sensitizing effects” at the hematopoietic protective dose of 100 ng/mouse. However, in agreement with the previous report, high dose IL-12 (1000 ng/mouse) resulted in more severe GI damage with shorter and broken villi when animals received 16 Gy radiation (FIG. 2b). These results clearly indicated that with the proper dose, it is feasible to obtain hematopoietic protection with IL-12 while avoiding GI toxicity.

### IL-12 Promotes Blood Cell Production After Lethal or Sublethal Dose Radiation

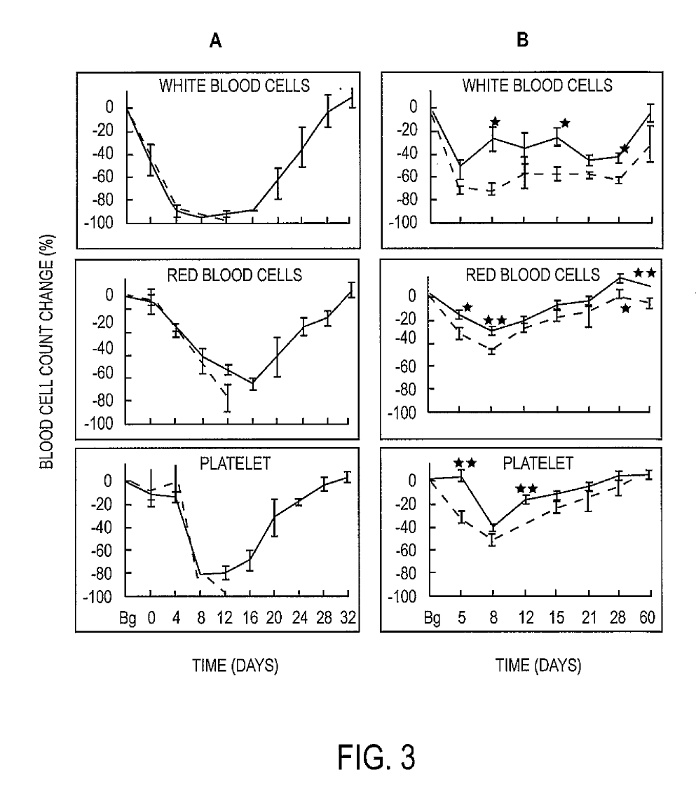

Since the bone marrow was protected by 100 ng/mouse of IL-12, we investigated whether or not IL-12 decreased the drop in peripheral blood cell count (PBCC). After 10 Gy of radiation, PBCC in both IL-12 treated and control mice dropped to a similar nadir point by day 12, then the PBCC of IL-12-treated mice began to recover while all control mice died (FIG. 3a). In sub-lethal radiation (5 Gy), IL-12 treatment significantly attenuated the decline of PBCC and accelerated the recovery of PBCC (FIG. 3b). In this experiment, the blood cell count baseline of mice in each group was recorded before the treatment. To present the data in a comparable manner, we normalized the blood cell count data collected at different times to the baseline data. It is worth noting that IL-12 promoted full lineage blood cell recovery, including white blood cell, red blood cell and platelets. This is different from currently used hematopoiesis stimulating factors which usually stimulate the recovery of one or a few lineages [33].

IL-12 Protects LTR HSC from Lethal Dose Radiation

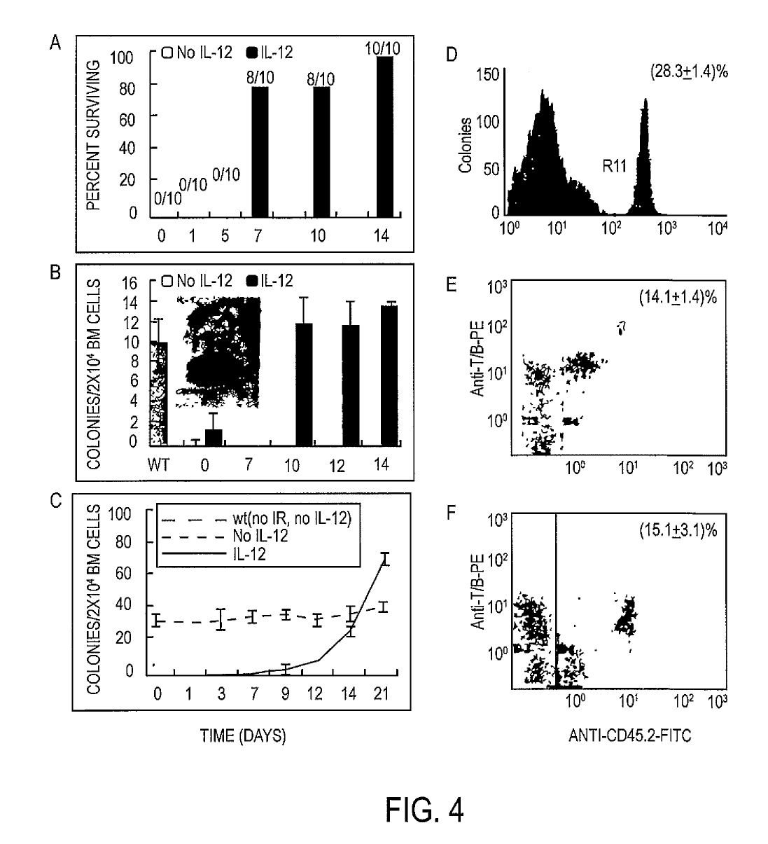

HSC are a group of heterogeneous cells which contain LTR HSC with full potential of self-renewal ability, short-term reconstituting (STR) HSC with limited self-renewal ability, and progenitor cells without self-renewal ability [32,34]. To determine which subsets of HSC survived the lethal dose radiation and replenished the blood system in IL-12 treated mice, we used different hematopoietic assays to examine the dynamic recovery of HSC activity; BMT to secondary lethally irradiated mice to test LTR HSC activities (FIG. 4a, d-f; colony-forming units-spleen12 (CFU-S₁₂) assay to test STR HSC/primitive progenitor activities [35] (FIG. 4b) and colony-forming cells (CFC) assay to test progenitor cells activities (FIG. 4c). The recovery of LTR HSC occurred at 7 days, followed by STR HSC/primitive progenitors at 10 days and progenitor cells at 14 days after radiation. This time sequence of detecting different cell activities was correlated to the HSC's maturation and differentiation pathway, from LTR HSC to STR HSC/primitive progenitor and then to progenitors. These data suggest that 10 Gy radiation destroyed both STR HSC/primitive progenitor and progenitor cells and the recovery of these cells derived from LTR HSC rescued by IL-12. Further more, when tested in BMT, the recovered LTR HSC could rescue lethally irradiated mice in a long-term and repopulate both myeloid and lymphoid cells (FIG. 4d-f). Although the spleen could be a hematopoietic organ in mice, it was not required for IL-12 mediated rescue: a prior splenectomy did not affect the radioprotective function of IL-12.

### IL-12 Treated Bone Marrow can Facilitate Donor Cell Engraftment in BMT

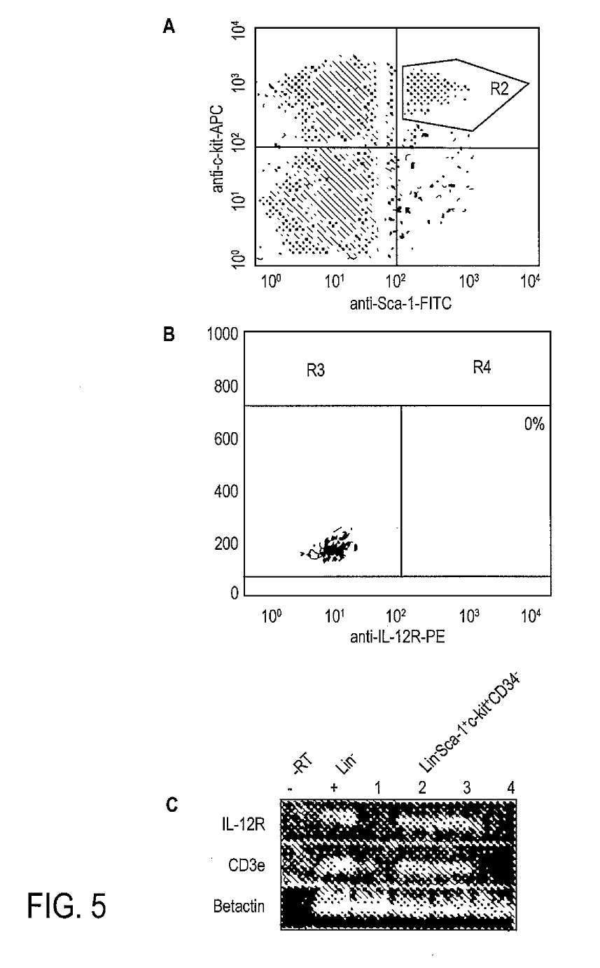

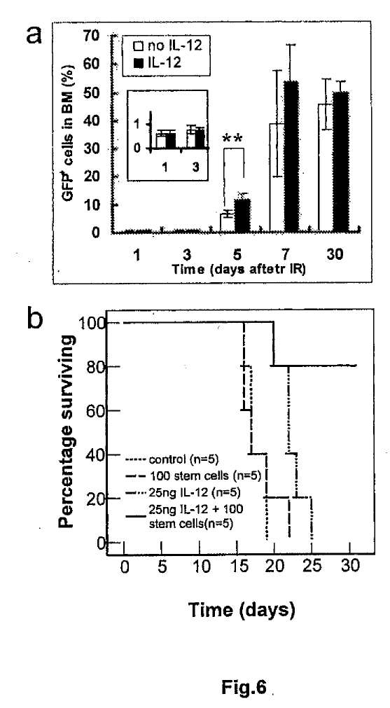

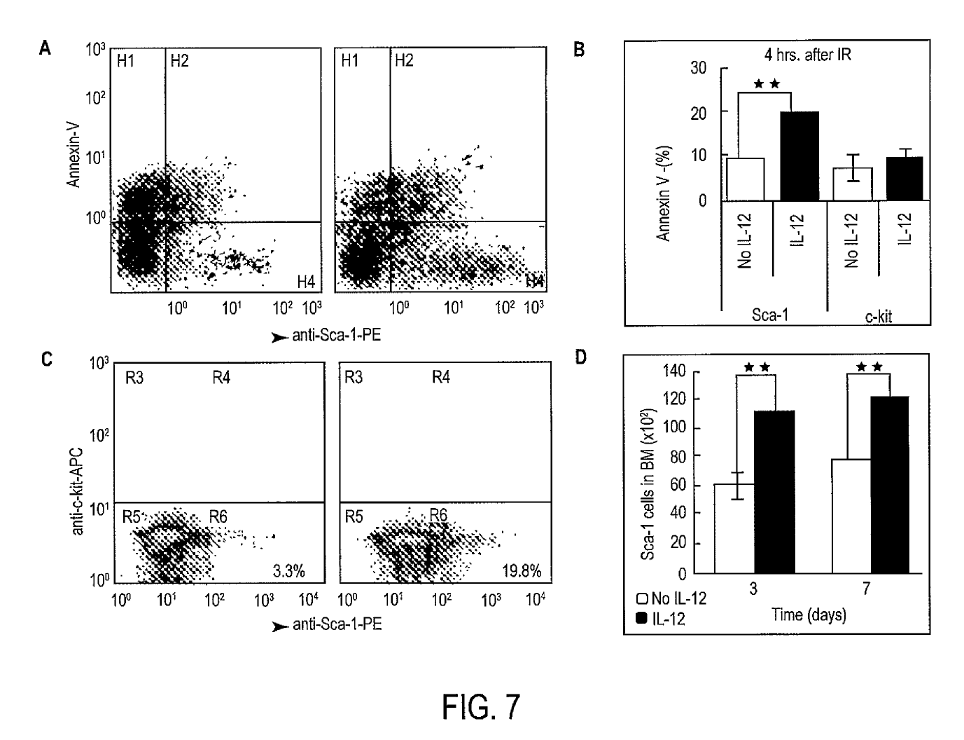

We next investigated if the IL-12 mediated radioprotection was via direct or indirect effect on LTR HSC. Using FACS analysis, we could not detect IL-12 receptor β2 chain expression in Lin⁻Sca-1⁺c-kit⁺ cells (FIG. 5a,b). Primers for IL-12 receptor, CD3e and β-actin were used in the analysis. Since RT-PCR is a very sensitive method to detect gene expression, CD3e primers were used as a control to determine if the sorted-samples are contaminated with T cells, which are known to express IL-12 receptor. As shown in FIG. 5c, 2 out of 4 examined samples showed no expression of IL-12 receptor and were CD3e negative. Another two samples were positive for both IL-12 receptor and CD3e, suggesting contamination of T cells in the sorted LTR HSC. These data indicate that there is no detectable IL-12 receptor expression on the LTR HSC. Also, these results suggest that IL-12 mediated radioprotection of LTR HSC is most likely to be an indirect effect, probably through the microenvironment.

The integrity of the bone marrow microenvironment is important for stem cell homing and proliferation. A change of microenvironment can affect homing and the proliferation abilities of donor cells in BMT. Using bone marrow cells from GFP (green fluorescent protein) transgenic mice as donor cells, we demonstrated that there was no significant difference in donor cells homing between IL-12-treated and control recipients since there was no difference in the number of donor cells at day 1 and 3 after BMT (FIG. 6a). However, by day 5 post BMT, the number of donor cells was significantly higher in IL-12-treated mice in comparison with control mice (p<0.01). This result suggests that the microenvironment of IL-12 treated bone marrow enhanced donor cell proliferation.

Next, we examined the influence of IL-12 treatment on stem cell transplantation. The experiment was to administer to lethally irradiated mice either a low dose of USC (100 Lin⁻Sca-1⁺c-kit⁺ cells) or a low dose of IL-12 (25 ng/mouse), or the two in combination. Neither low dose stem cell transplantation nor low dose of IL-12 administration alone could rescue lethally irradiated animals. However, the combination rescued 80% of the recipient mice (FIG. 6b). Since IL-12 did not improve the homing of donor cells, these results suggest that the bone marrow microenvironment of IL-12 treated mice was more resistant to radiation and could thus facilitate the donor stem cell proliferation and engraftment.

IL-12 Protects Sca-1⁺ Bone Marrow Cells from Lethal Dose Radiation

Radiation induced apoptosis is the major cause of tissue damage [36,37]. To identify which cell subset was protected by IL-12, we performed FACS analysis of cells co-strained for Annexin V and cell type specific markers. Among all the cell surface markers examined (Sca-1, c-kit, CD31, CD105 and Alkaline Phosphotase), we found that after irradiation, the Annexin V⁻/Sca-1⁺ cell number was significantly higher in IL-12 treated animals than in control animals (FIG. 7a&b, p<0.05). We then followed the dynamic change of the cellularity at different times post radiation. The Sca-1⁺ population remained at a significantly higher level in IL-12 treated animals than in control animals (FIG. 7d, p<0.001 for IL-12 treated vs. control animals at day 3 and 7 post radiation respectively). On the other hand, there was no significant difference in AnnexinV⁻/c-kit⁺ cell number between IL-12 treated and control animals after irradiation (FIG. 7b). Further more, as early as 24 hrs post irradiation, there were no detectable Lin⁻Sca-1⁺c-kit⁺ cells in both IL-12 treated and control animals (FIG. 7c). It has been reported that both long and short-term HSC reside in Lin⁻Sca-1⁺c-kit⁺ cells [32,38]. However, the depletion of Lin⁻Sca-1⁺c-kit⁺ cells in IL-12 treated mice suggest that the recovery of the LTR HSC in rescued animals might be from cells without detectable level of c-kit expression.

### Discussion

It this study, we showed that IL-12 could effectively protect animals from lethal radiation and attenuate the decline of the blood cell count after sublethal irradiation without sensitizing the GI system. Further more, we also demonstrated that the replenished blood system after irradiation originated from the LTR HSC. Since there was no detectable IL-12 receptor expression on the LTR HSC, our results suggest that the protective effect from IL-12 may be on the microenvironment or un-identified HSC population which can not be identified with the cell surface markers used in this study (Lin⁻Sca-1⁺c-kit⁺ CD34⁻). In the BMT experiments, we showed that although the homing efficiency of IL-12 treated or control bone marrow was similar, the donor cells in IL-12 treated bone marrow proliferated more rapidly compared with the cells in control bone marrow, suggesting that the microenvironment of IL-12 treated bone marrow could facilitate the stem cell engraftment.

HSC and its microenvironment in bone marrow are composed of heterogeneous cellular components, which form a precisely organized structural architecture for a complicated structural/functional relationship. Radio-sensitivity is different among various cell types. The LTR HSC is less sensitive to radiation since they are quiescent cells in the Go/G1 phase. It is possible that the lethal dose used in this study did not destroy the LTR HSC, instead, it destroyed cells in microenvironment which are important in supporting and regulating the LTR HSC. Since there was no detectable IL-12 receptor expression in the LTR HSC, it is likely that IL-12 plays its role by protecting the cells in microenvironment that facilitates the recovery of endogenous LTR HSC and the engraftment of HSC in the BMT. The recovered LTR HSC can give rise to a full lineage cell recovery. IL-12 mediated hematopoietic system protection can be a direct effect on the target cells or indirect effect by stimulating the production of other factors that then protects the LTR HSC and/or cells in microenvironment.

There were significantly more Sca-1⁺ cells in IL-12 treated animals compared to control animals. Although known as a surface marker for HSC, Sca-1 expression has also been reported on the surface of mesenchymal stem cells, endothelial progenitor and osteoblast progenitor cells [39-42]. All are important cellular components of the microenvironment [5,6,8-10, 43, 44]. The protected Sca-1⁺ cells may be a mixture of several different cell types that function together to restore the microenvironment, which then stimulates the hematopoiesis. Another possibility is that the protected Sca-1⁺ cells have the property of “multipotent adult progenitor cells” [45] which may give rise to cells that rebuild the BM microenvironment, as well as HSC.

To achieve effective protection of hematopoiesis, IL-12 needs to be administrated in a limited time window relative to the time of irradiation: either 24 hrs before or 1 hr after radiation. In previous studies, most of the factors had to be administrated 24 hrs before irradiation [11-15]. Few reports showed protective effects when factors were given after irradiation [17, 18, 46]. When given 24 hours before radiation, the protective factors may increase the radio-resistance of the target cells. It was proposed that cells at late S-phase of cell cycle are more resistant to radiation [47,48]. When given immediately after irradiation, the factors may function via an anti-apoptosis mechanism by stopping the radiation triggered apoptotic pathway [17,18]. Since IL-12 is effective when administrated both before and after radiation, it may function at multiple levels.

It is commonly accepted that HSC reside in the Lin⁻Sca-1⁺c-kit⁺ population. The lack of Lin⁻Sca-1⁺c-kit⁺ cells after radiation in both IL-12 treated and control animals raise the question of which cell type repopulates the blood system. A HSC subset with positive Sca-1 expression but negative c-kit has been reported [49]. But, these cells can only give delayed reconstitution and cannot rescue lethally irradiated animals. The observed reconstitution in IL-12 protected animals may not be from these cells. It is possible that irradiation down regulates c-kit expression in HSC as it has been reported that cytotoxic agents down regulate the c-kit expression [50].

In summary, this study demonstrated that IL-12 can effectively protect the bone marrow from irradiation and promote the recovery of hematopoiesis. IL-12 can facilitate the donor cell engraftment in BMT. Although there have been reported adverse effects on the GI system from IL-12 mediated IFN-y release, we demonstrated that proper dose control can avoid the onset of this side effect. This study raises the possibility of using IL-12 as an adjuvant therapeutic agent to enhance the recovery of endogenous HSC after radio-/chemo-therapy or to facilitate the engraftment of stem cells in BMT.

Obviously, many modifications and variation of the invention as hereinbefore set forth can be made without departing from the spirit and scope thereof and therefore only such limitations should be imposed as are indicated by the appended claims.

All patent and literature references cited in the present specification are hereby incorporated by reference in their entirety.

### REFERENCES

1. Lemischka IR (1997) Microenvironmental regulation of hematopoietic stem cells. Stem Cells 15 Suppl 1:63.
2. Zhu J, Emerson S G (2004) A new bone to pick: osteoblasts and the haematopoietic stem-cell niche. Bioessays 26:595.
3. Moore K A (2004) Recent advances in defining the hematopoietic stem cell niche. Curr Opin Hematol 11:107.
4. Taichman R S (2005) Blood and bone: two tissues whose fates are intertwined to create the hematopoietic stem-cell niche. Blood 105:2631.
5. Arai F, Hirao A, Ohmura M, Sato H, Matsuoka S, Takubo K, Ito K, Koh G Y, Suda T (2004) Tie2/angiopoietin-1 signaling regulates hematopoietic stem cell quiescence in the bone marrow niche. Cell 118:149.
6. Calvi L M, Adams G B, Weibrecht K W, Weber J M, Olson D P, Knight M C, Martin R P, Schipani E, Divieti P, Bringhurst F R, Milner L A, Kronenberg H M, Scadden D T (2003) Osteoblastic cells regulate the haematopoietic stem cell niche. Nature 425:841.
7. Taichman R S, Emerson S G (1998) The Role of Osteoblasts in the Hematopoietic Microenvironment. Stem Cells 16:7.
8. Zhang J, Niu C, Ye L, Huang H, He X, Tong W G, Ross J, Haug J, Johnson T, Feng J Q, Harris S, Wiedemann L M, Mishina Y, Li L (2003) Identification of the haematopoietic stem cell niche and control of the niche size. Nature 425:836.
9. Kiel M J, Yilmaz O H, Iwashita T, Yilmaz O H, Terhorst C, Morrison S J (2005) SLAM Family Receptors Distinguish Hematopoietic Stem and Progenitor Cells and Reveal Endothelial Niches for Stem Cells. Cell 121:1109.
10. Kopp H-G, Avecilla S T, Hooper A T, Shmelkov S V, Ramos C A, Zhang F, Rafii S (2005) Tie2 activation contributes to hemangiogenic regeneration after myelosuppression. Blood 106:505.
11. Zsebo K M, Smith K A, Hartley C A, Greenblatt M, Cooke K, Rich W, McNiece I K (1992) Radioprotection of mice by recombinant rat stem cell factor. Proc Natl Acad Sci USA 89:9464.
12. Maddens S, Charruyer A, Plo I, Dubreuil P, Berger S, Salles B, Laurent G, Jaffrezou J-P (2002) Kit signaling inhibits the sphingomyelin-ceramide pathway through PLCgamma 1: implication in stem cell factor radioprotective effect. Blood 100:1294.
13. Dalmau S R, Freitas C S, Tabak D G (1993) Interleukin-1 and tumor necrosis factor-alpha as radio- and chemoprotectors of bone marrow. Bone Marrow Transplant 12:551.
14. Gratwohl A, John L, Baldomero H, Roth J, Tichelli A, Nissen C, Lyman S D, Wodnar-Filipowicz A (1998) FLT-3 ligand provides hematopoietic protection from total body irradiation in rabbits. Blood 92:765.
15. Neta R, Stiefel S M, Finkelman F, Herrmann S, Ali N (1994) IL-12 protects bone marrow from and sensitizes intestinal tract to ionizing radiation. J Immunol 153:4230.
16. Drouet M, Mourcin F, Grenier N, Leroux V, Denis J, Mayol J F, Thullier P, Lataillade J J, Herodin F (2004) Single administration of stem cell factor, FLT-3 ligand, megakaryocyte growth and development factor, and interleukin-3 in combination soon after irradiation prevents nonhuman primates from myelosuppression: long-term follow-up of hematopoiesis. Blood 103:878.
17. Herodin F, Bourin P, Mayol J F, Lataillade J J, Drouet M (2003) Short-term injection of antiapoptotic cytokine combinations soon after lethal gamma irradiation promotes survival. Blood 101:2609.
18. Neelis K J, Visser T P, Dimjati W, Thomas G R, Fielder P J, Bloedow D, Eaton D L, Wagemaker G (1998) A single dose of thrombopoietin shortly after myelosuppressive total body irradiation prevents pancytopenia in mice by promoting short-term multilineage spleen-repopulating cells at the transient expense of bone marrow-repopulating cells. Blood 92:1586.
19. Paris F, Fuks Z, Kang A, Capodieci P, Juan G, Ehleiter D, Haimovitz-Friedman A, Cordon-Cardo C, Kolesnick R (2001) Endothelial apoptosis as the primary lesion initiating intestinal radiation damage in mice. Science 293:293.
20. Kobayashi M, Fitz L, Ryan M, Hewick R, Clark S, Chan S, Loudon R, Sherman F, Perussia B, Trinchieri G (1989) Identification and purification of natural killer cell stimulatory factor (NKSF), a cytokine with multiple biologic effects on human lymphocytes. J Exp Med 170:827.
21. Manetti R, Parronchi P, Giudizi M, Piccinni M, Maggi E, Trinchieri G, Romagnani S (1993) Natural killer cell stimulatory factor (interleukin 12 [L-12]) induces T helper type 1 (Th1)-specific immune responses and inhibits the development of IL-4-producing Th cells. J Exp Med 177:1199.
22. Zeh H J, 3rd, Hurd S, Storkus W J, Lotze M T (1993) Interleukin-12 promotes the proliferation and cytolytic maturation of immune effectors: implications for the immunotherapy of cancer. J Immunother 14:155.
23. Ploemacher R E, van Soest P L, Voorwinden H, Boudewijn A (1993) Interleukin-12 synergizes with interleukin-3 and steel factor to enhance recovery of murine hemopoietic stem cells in liquid culture. Leukemia 7:1381.
24. Hirayama F, Katayama N, Neben S, Donaldson D, Nickbarg E, Clark S, Ogawa M (1994) Synergistic interaction between interleukin-12 and steel factor in support of proliferation of murine lymphohematopoietic progenitors in culture. Blood 83:92.
25. Kang H, Mansel R E, Jiang W G (2005) Genetic manipulation of stromal cell-derived factor-1 attests the pivotal role of the autocrine SDF-1-CXCR4 pathway in the aggressiveness of breast cancer cells. Int J Oncol 26:1429.
26. Landuzzi L, De Giovanni C, Nicoletti G, Rossi I, Ricci C, Astolfi A, Scopece L, Scotlandi K, Serra M, Bagnara G P, Nanni P, Lollini P L (2000) The metastatic ability of Ewing's sarcoma cells is modulated by stem cell factor and by its receptor c-kit. Am J Pathol 157:2123.
27. Maulik G, Bharti A, Khan E, Broderick R J, Kijima T, Salgia R (2004) Modulation of c-Kit/SCF pathway leads to alterations in topoisomerase-I activity in small cell lung cancer. J Environ Pathol Toxicol Oncol 23:237.
28. Porcile C, Bajetto A, Barbieri F, Barbero S, Bonavia R, Biglieri M, Pirani P, Florio T, Schettini G (2005) Stromal cell-derived factor-1alpha (SDF-1alpha/CXCL12) stimulates ovarian cancer cell growth through the EGF receptor transactivation. Exp Cell Res
29. Colombo M P, Trinchieri G (2002) Interleukin-12 in anti-tumor immunity and immunotherapy. Cytokine Growth Factor Rev 13:155.
30. Voest E E, Kenyon B M, O'Reilly M S, Truitt G, D'Amato R J, Folkman J (1995) Inhibition of angiogenesis in vivo by interleukin 12. J Natl Cancer Inst 87:581.
31. Yao L, Pike S E, Setsuda J, Parekh J, Gupta G, Raffeld M, Jaffe E S, Tosato G (2000) Effective targeting of tumor vasculature by the angiogenesis inhibitors vasostatin and interleukin-12. Blood 96:1900.
32. Zhao Y, Lin Y, Zhan Y, Yang G, Louie J, Harrison D E, Anderson W F (2000) Murine hematopoietic stem cell characterization and its regulation in BM transplantation. Blood 96:3016.
33. Dempke W, Von Poblozki A, Grothey A, Schmoll H J (2000) Human hematopoietic growth factors: old lessons and new perspectives. Anticancer Res 20:5155.
34. Weissman I L, Anderson D J, Gage F (2001) Stem and progenitor cells: origins, phenotypes, lineage commitments, and transdifferentiations. Annu Rev Cell Dev Biol 17:387.
35. Till J E, Mc C E (1961) A direct measurement of the radiation sensitivity of normal mouse bone marrow cells. Radiat Res 14:213.
36. Verheij M, Bartelink H (2000) Radiation-induced apoptosis. Cell & Tissue Research 301:133.
37. Held K D (1997) Radiation-induced apoptosis and its relationship to loss of clonogenic survival. Apoptosis 2:265.
38. Osawa M, Hanada K, Hamada H, Nakauchi H (1996) Long-term lymphohematopoietic reconstitution by a single CD34-low/negative hematopoietic stem cell. Science 273:242.
39. Baddoo M, Hill K, Wilkinson R, Gaupp D, Hughes C, Kopen G C, Phinney D G (2003) Characterization of mesenchymal stem cells isolated from murine bone marrow by negative selection. J Cell Biochem 89:1235.
40. Kotton D N, Summer R S, Sun X, Ma B Y, Fine A (2003) Stem cell antigen-1 expression in the pulmonary vascular endothelium. Am J Physiol Lung Cell Mol Physiol 284:L990
41. Luna G, Paez J, Cardier J E (2004) Expression of the hematopoietic stem cell antigen Sca-1 (LY-6A/E) in liver sinusoidal endothelial cells: possible function of Sca-1 in endothelial cells. Stem Cells Dev 13:528.
42. Shaw J P, Basch R, Shamamian P (2004) Hematopoietic stem cells and endothelial cell precursors express Tie-2, CD31 and CD45. Blood Cells Mol Dis 32:168.
43. Balduino A, Hurtado S P, Frazao P, Takiya C M, Alves L M, Nasciutti L E, El-Cheikh M C, Borojevic R (2005) Bone marrow subendosteal microenvironment harbours functionally distinct haemosupportive stromal cell populations. Cell Tissue Res 319:255.
44. Stier S, Ko Y, Forkert R, Lutz C, Neuhaus T, Grunewald E, Cheng T, Dombkowski D, Calvi L M, Rittling S R, Scadden D T (2005) Osteopontin is a hematopoietic stem cell niche component that negatively regulates stem cell pool size. J Exp Med 201:1781.
45. Jiang Y, Jahagirdar B N, Reinhardt R L, Schwartz R E, Keene C D, Ortiz-Gonzalez X R, Reyes M, Lenvik T, Lund T, Blackstad M, Du J, Aldrich S, Lisberg A, Low W C, Largaespada D A, Verfaillie C M (2002) Pluripotency of mesenchymal stem cells derived from adult marrow. Nature 418:41.
46. Zhao Y, Zhan Y, Burke K A, Anderson W F (2005) Soluble factor(s) from bone marrow cells can rescue lethally irradiated mice by protecting endogenous hematopoietic stem cells. Exp Hematol 33:428.
47. Neta R (1988) Role of cytokines in radioprotection. Pharmacology & Therapeutics 39:261.
48. Neta R (1997) Modulation with cytokines of radiation injury: suggested mechanisms of action. Environ Health Perspect 105 Suppl 6:1463.
49. Ortiz M, Wine J W, Lohrey N, Ruscetti F W, Spence S E, Keller J R (1999) Functional Characterization of a Novel Hematopoietic Stem Cell and Its Place in the c-Kit Maturation Pathway in Bone Marrow Cell Development. Immunity 10:173.
50. Randall T D, Weissman I L (1997) Phenotypic and Functional Changes Induced at the Clonal Level in Hematopoietic Stem Cells After 5-Fluorouracil Treatment, Blood 89:3596.

What is claimed is:

1. A composition comprising IL-12 that promotes the recovery of hematopoeisis to bone marrow cells that have been damaged by radiation and/or chemotherapy.

2. A composition comprising IL-12 that protects bone marrow from myelosuppression that have been damaged by radiation and/or chemotherapy.

3. A composition comprising IL-12 that protects hematopoeisis in a subject that has been treated with radiation and/or chemotherapy.

4. A composition comprising IL-12 that protects the bone marrow microenvironment from damage caused by radiation and/or chemotherapy.

5. A composition comprising IL-12 that facilitates the engraftment of hematopoietic stem cell transplantation in a subject.

6. A method of promoting the recovery of hematopoeisis in bone marrow cells that have been treated with radiation and/or chemotherapy, said method comprising administering a composition comprising IL-12 to a subject before said subject is treated with radiation and/or chemotherapy.

7. The method according to claim 6, wherein said compositions is administered 24 hours before treatment with radiation and/or chemotherapy.

8. The method according to claim 6, wherein said compositions is administered 1 hour after treatment with radiation and/or chemotherapy.

9. The method according to claim 6, further comprising administering said composition after treatment with said radiation and/or chemotherapy.

10. A method of protecting bone marrow from myelosuppression comprising administering a composition comprising IL-12 to a subject before said subject is treated with radiation and/or chemotherapy.

11. The method according to claim 10, wherein said compositions is administered 24 hours before treatment with radiation and/or chemotherapy.

12. The method according to claim 10, wherein said compositions is administered 1 hour after treatment with radiation and/or chemotherapy.

13. The method according to claim 10, further comprising administering said composition after treatment with said radiation and/or chemotherapy.

14. A method of protecting hematopoeisis in a subject comprising administering a composition comprising IL-12 to a subject before said subject is treated with radiation and/or chemotherapy.

15. The method according to claim 14, wherein said compositions is administered 24 hours before treatment with radiation and/or chemotherapy.

16. The method according to claim 14, wherein said compositions is administered 1 hour after treatment with radiation and/or chemotherapy.

17. The method according to claim 14, further comprising administering said composition after treatment with said radiation and/or chemotherapy.

18. A method of protecting the bone marrow microenvironment from damage caused by radiation and/or chemotherapy comprising administering a composition comprising IL-12 to a subject before said subject is treated with radiation and/or chemotherapy.

19. The method according to claim 18, wherein said compositions is administered 24 hours before treatment with radiation and/or chemotherapy.

20. The method according to claim 18, wherein said compositions is administered 1 hour after treatment with radiation and/or chemotherapy.

21. The method according to claim 18, further comprising administering said composition after treatment with said radiation and/or chemotherapy.

22. A method of facilitating the engraftment of hematopoietic stem cell transplantation in a subject comprising administering composition comprising IL-12 to said subject before hematopoietic stem cell transplantation.

23. The method according to claim 6, wherein said compositions is administered 24 hours before transplantation.

24. The method according to claim 6, wherein said compositions is administered 1 hour after transplantation.

25. The method according to claim 6, further comprising administering said composition after transplantation.


SEQUENCE LISTING (6 sequences)

SEQ ID NO: 1 - DNA, 20 bp, Artificial
  Primer
gcaaacagcacttgggtaaa

SEQ ID NO: 2 - DNA, 21 bp, Artificial
  Primer
ttcctgtagcttgtggattgg

SEQ ID NO: 3 - DNA, 20 bp, Artificial
  Primer
gatgcggtggaacactttct

SEQ ID NO: 4 - DNA, 20 bp, Artificial
  Primer
actgtcctcgacttccgaga

SEQ ID NO: 5 - DNA, 20 bp, Artificial
  Primer
actgggacgacatggagaag

SEQ ID NO: 6 - DNA, 20 bp, Artificial
  Primer
accagaggcatacagggaca